(19) United States
(12) Patent Application Publication
(10) Pub. No.: US 2026/0188492 A1
(43) Pub. Date: Jul. 2, 2026

(54) Systems and Methods for Identifying Protein-Losing Nephropathy

(71) Applicant: IDEXX Laboratories, Inc., Westbrook, ME (US)

(72) Inventor: Mary Alexis Seguin, North Freedom, WI (US)

(73) Assignee: IDEXX Laboratories, Inc., Westbrook, ME (US)

(21) Appl. No.: 19/397,000

(22) Filed: Nov. 21, 2025

Related U.S. Application Data
(60) Provisional application No. 63/740,452, filed on Dec. 31, 2024.

(51) Int. Cl. G16H 50/20; G16B 40/10; G16B 40/20

(52) U.S. Cl. CPC G16H 50/20; G16B 40/10; G16B 40/20

(57) ABSTRACT

A method for identifying the risk of protein losing nephropathy (PLN) includes receiving new patient data, receiving a machine-learning based diagnostic model and a knowledge based diagnostic model, determining whether the machine-learning based diagnostic model indicates a risk for PLN for the new patient data, and in a case where the machine-learning based diagnostic model indicates a risk for PLN for the new patient data, assessing the risk of PLN using the knowledge based diagnostic model.

### CROSS-REFERENCE TO RELATED APPLICATIONS

This application claims the benefit of co-pending U.S. Provisional Patent Application Serial No. 63/740,452, filed December 31, 2024 which is hereby incorporated by reference in its entirety.

### FIELD

The present disclosure generally relates to systems and methods for veterinary applications, and more specifically relates to systems and methods for identifying risk of protein-losing nephropathy (PLN).

### BACKGROUND

PLN, also referred to as glomerulonephritis, is a kidney disease in which damage to the glomeruli results in leakage of protein into the urine. The most common cause of PLN is chronic inflammation or infection elsewhere in the body, although familial PLN and autoimmune PLN may also occur. PLN is common in dogs, and incidence of PLN may be as high as 50%.

Early in the course of the disease, clinical signs are typically absent, and detection is based on detection of protein in the urine on a urinalysis. Currently, interpretation of protein on a chemical urinalysis requires consideration of urine concentration and ruling out pre- and post-renal causes of proteinuria. Renal proteinuria is confirmed by determining a urine protein/creatinine ratio (UPC) in the urine sample.

Left untreated, serum albumin levels begin to drop. Protein leaking through the glomeruli may accumulate in the tubules, resulting in secondary tubular damage and the development of chronic kidney disease (CKD). Eventually nephrotic syndrome may develop in end-stage PLN. This is a syndrome where low serum albumin results in the buildup of fluid in the abdomen (ascites) or tissues (edema), and is also accompanied by high cholesterol, systemic hypertension and hypercoagulability. Pulmonary thromboembolism is a potential complication of the hypercoagulable state. Early diagnosis and intervention for PLN may slow the progression of the disease and potentially prevent CKD and nephrotic syndrome from developing. However, cases may be missed in the early stages of disease if a urinalysis is not performed.

Currently only 10-20% of CBC/Chemistry bloodwork in the United States is accompanied by a UA. Accordingly, many veterinary subjects at risk of PLN are not identified for early intervention, leading to suboptimal health outcomes.

### BRIEF DESCRIPTION OF THE DRAWINGS

Various aspects and features of the present disclosure are described hereinbelow with reference to the drawings wherein:

FIG. 1 shows a computing device system, according to one or more embodiments described herein;

FIG. 2 shows another computing device system, according to one or more embodiments described herein;

FIG. 3 shows a hematology analyzer, according to one or more embodiments described herein;

FIG. 4 shows a controller of the hematology analyzer of FIG. 3, according to one or more embodiments described herein;

FIG. 5 shows a block diagram of a diagnostic system for identifying PLN, according to one or more embodiments described herein;

FIG. 6 shows a flowchart of a machine learning PLN identification method, according to one or more embodiments described herein;

FIG. 7 shows a flowchart of a method of generating machine learning models for identifying PLN, according to one or more embodiments described herein; and

FIG. 8 shows a flowchart of an interactive machine learning based PLN identification method, according to one or more embodiments described herein.

### DETAILED DESCRIPTION

Various embodiments now will be described more fully hereinafter with reference to the accompanying drawings, in which some but not all embodiments are shown. The term “or” is used herein in both the alternative and conjunctive sense, unless otherwise indicated. In addition, unless otherwise explicitly noted or required by context, the word “set” is intended to mean one or more. For example, the phrase, “a set of objects” means one or more of the objects. The terms “illustrative” and “exemplary” are used to be examples with no indication of quality level. And terms are used both in the singular and plural forms interchangeably. Like numbers refer to like elements throughout.

In some aspects of the disclosure, the computer systems described herein execute methods for implementing machine learning models that identify PLN in animals, such as cats and dogs. It should be noted that the aspects or embodiments of the present disclosure are not limited to these or any other examples provided herein, which are referred to for purposes of illustration only.

In this regard, in the descriptions herein, certain specific details are set forth to provide a thorough understanding of various aspects of the disclosure. However, one skilled in the art will understand that the embodiments described herein may be practiced at a more general level without one or more of these details. In other instances, well-known structures have not been shown or described in detail to avoid unnecessarily obscuring descriptions of various aspects of the disclosure.

Any reference throughout this specification to one “aspect” or “embodiment,” an “aspect” or “embodiment,” an example “aspect” or “embodiment,” an illustrated “aspect” or “embodiment,” a particular “aspect” or “embodiment,” or the like means that a particular feature, structure, or characteristic described in connection with the aspect or embodiment is included in one or more aspects or embodiments. Thus, any appearance of the phrase in one “aspect” or “embodiment,” in an “aspect” or “embodiment,” in an example “aspect” or “embodiment,” in this illustrated “aspect” or “embodiment,” in this particular “aspect” or “embodiment,” or the like in this specification is not necessarily all referring to one aspect or embodiment or a same aspect or embodiment. Furthermore, the particular features, structures or characteristics of different aspects or embodiments of the disclosure may be combined in any suitable manner to form one or more other aspects or embodiments of the disclosure. Further, the term aspect or embodiment may be used interchangeably.

In the following description, some aspects of the disclosure may be implemented at least in part by a data processing device system configured by a software program. Such a program may equivalently be implemented as multiple programs, and some or all of such software program(s) may be equivalently constructed in hardware.

Further, the phrase “at least” is or may be used herein at times merely to emphasize the possibility that other elements may exist beside those explicitly listed. However, unless otherwise explicitly noted (such as by the use of the term “only”) or required by context, non-usage herein of the phrase “at least” nonetheless includes the possibility that other elements may exist besides those explicitly listed. For example, the phrase, “based at least on A” includes A as well as the possibility of one or more other additional elements besides A. In the same manner, the phrase, “based on A” includes A, as well as the possibility of one or more other additional elements besides A. However, the phrase, “based only on A” includes only A. Similarly, the phrase “configured at least to A” includes a configuration to perform A, as well as the possibility of one or more other additional actions besides A. In the same manner, the phrase “configured to A” includes a configuration to perform A, as well as the possibility of one or more other additional actions besides A. However, the phrase, “configured only to A” means a configuration to perform only A.

The word “device,” the word “machine,” the word “system,” and the phrase “device system” all are intended to include one or more physical devices or sub-devices (e.g., pieces of equipment) that interact to perform one or more functions, regardless of whether such devices or sub-devices are located within a same housing or different housings. However, it may be explicitly specified according to various aspects of the disclosure that a device or machine or device system resides entirely within a same housing to exclude aspects of the disclosure where the respective device, machine, system, or device system resides across different housings. The word “device” may equivalently be referred to as a “device system” in some aspects of the disclosure.

The phrase “derivative thereof” and the like is or may be used herein at times in the context of a derivative of data or information merely to emphasize the possibility that such data or information may be modified or subject to one or more operations. For example, if a device generates first data for display, the process of converting the generated first data into a format capable of being displayed may alter the first data. This altered form of the first data may be considered a derivative of the first data. For instance, the first data may be a one-dimensional array of numbers, but the display of the first data may be a color-coded bar chart representing the numbers in the array. For another example, if the above-mentioned first data is transmitted over a network, the process of converting the first data into a format acceptable for network transmission or understanding by a receiving device may alter the first data. As before, this altered form of the first data may be considered a derivative of the first data. For yet another example, generated first data may undergo a mathematical operation, a scaling, or a combining with other data to generate other data that may be considered derived from the first data. In this regard, it can be seen that data is commonly changing in form or being combined with other data throughout its movement through one or more data processing device systems, and any reference to information or data herein is intended to include these and like changes, regardless of whether or not the phrase “derivative thereof” or the like is used in reference to the information or data, unless otherwise required by context. As indicated above, usage of the phrase “or a derivative thereof” or the like merely emphasizes the possibility of such changes. Accordingly, the addition of or deletion of the phrase “or a derivative thereof” or the like should have no impact on the interpretation of the respective data or information. For example, the above-discussed color-coded bar chart may be considered a derivative of the respective first data or may be considered the respective first data itself.

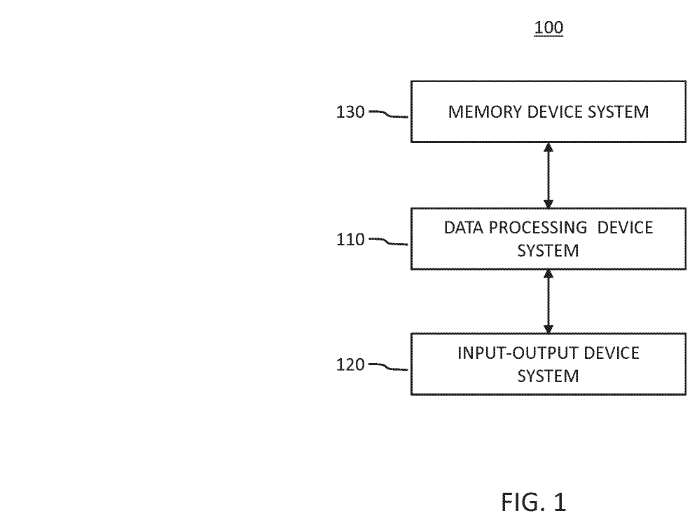

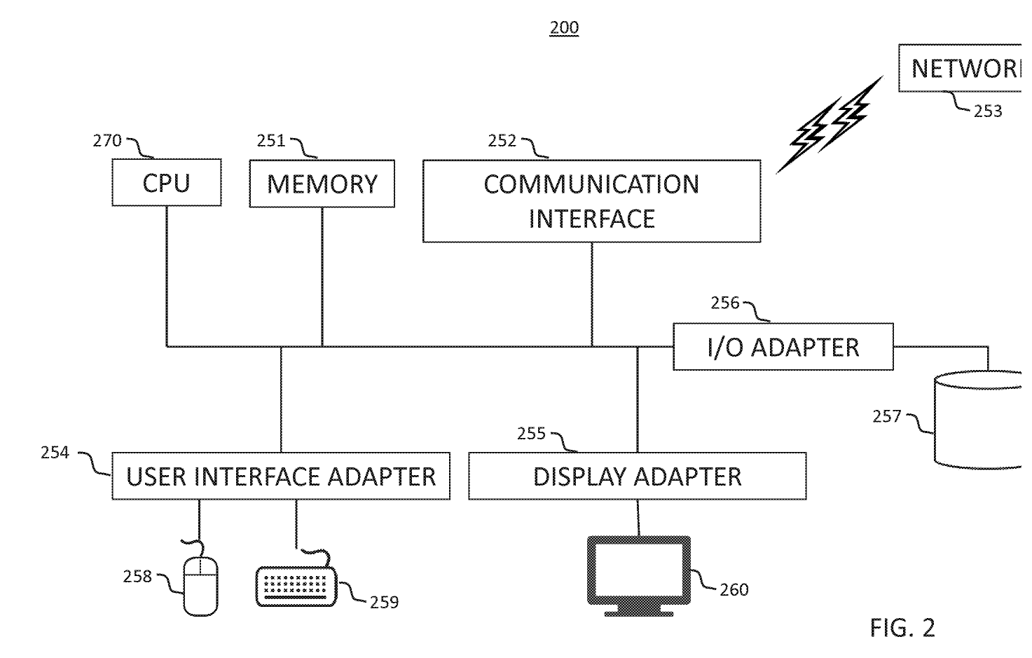

The term “program” in this disclosure should be interpreted to include one or more programs including a set of instructions or modules that may be executed by one or more components in a system, such as a controller system or data processing device system, to cause the system to perform one or more operations. The set of instructions or modules may be stored by any kind of memory device, such as those described subsequently with respect to the memory 130, 251, or both, shown in FIGS. 1 and 2, respectively. In addition, this disclosure may describe or similarly describe that the instructions or modules of a program are configured to cause the performance of an action. The phrase “configured to” in this context is intended to include at least

(a) instructions or modules that are presently in a form executable by one or more data processing devices to cause performance of the action (e.g., in the case where the instructions or modules are in a compiled and unencrypted form ready for execution), and (b) instructions or modules that are presently in a form not executable by the one or more data processing devices, but could be translated into the form executable by the one or more data processing devices to cause performance of the action (e.g., in the case where the instructions or modules are encrypted in a non-executable manner, but through performance of a decryption process, would be translated into a form ready for execution). Such descriptions should be deemed to be equivalent to describing that the instructions or modules are configured to cause the performance of the action. The word “module” may be defined as a set of instructions. The word “program” and the word “module” may each be interpreted to include multiple sub-programs or multiple sub-modules, respectively. In this regard, reference to a program or a module may be considered to refer to multiple programs or multiple modules.

Further, it is understood that information or data may be operated upon, manipulated, or converted into different forms as it moves through various devices or workflows. In this regard, unless otherwise explicitly noted or required by context, it is intended that any reference herein to information or data includes modifications to that information or data. For example, “data X” may be encrypted for transmission, and a reference to “data X” is intended to include both its encrypted and unencrypted forms, unless otherwise required or indicated by context. However, non-usage of the phrase “or a derivative thereof” or the like nonetheless includes derivatives or modifications of information or data just as usage of such a phrase does, as such a phrase, when used, is merely used for emphasis.

Further, the phrase “graphical representation” used herein is intended to include a visual representation presented via a display device system and may include computer-generated text, graphics, animations, or one or more combinations thereof, which may include one or more visual representations originally generated, at least in part, by an image-capture device.

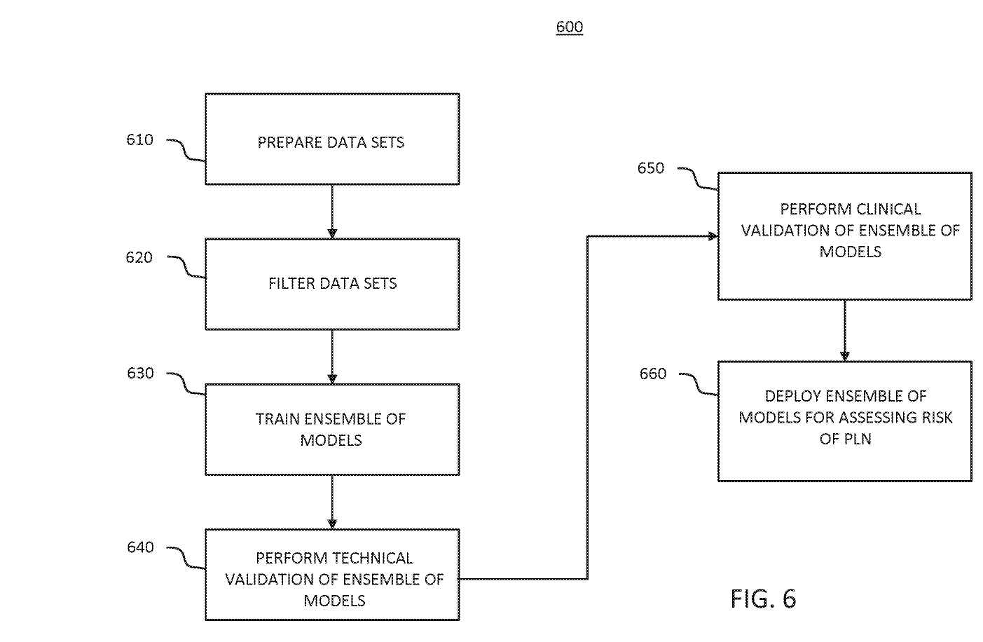

Further still, example methods are described herein with respect to FIGS. 6-8. Such figures are described to include blocks associated with computer-executable instructions. It should be noted that the respective instructions associated with any such blocks herein need not be separate instructions and may be combined with other instructions to form a combined instruction set. The same set of instructions may be associated with more than one block. In this regard, the block arrangements shown in method FIGS. 6-8 herein are not limited to an actual structure of any program or set of instructions or required ordering of method tasks, and such methods as depicted in FIGS. 6-8, according to some aspects of the disclosure, merely illustrate the tasks that instructions are configured to perform, for example upon execution by a data processing device system in conjunction with interactions with one or more other devices or device systems.

FIG. 1 schematically illustrates a system 100 according to some aspects of the disclosure. In some aspects of the disclosure, the system 100 may be a computing device system 200 (as shown in FIG. 2). In some aspects of the disclosure, the system 100 includes a data processing device system 110, an input-output device system 120, and a processor-accessible memory device system 130. The processor-accessible memory device system 130 and the input-output device system 120 are communicatively connected to the data processing device system 110.

The data processing device system 110 includes one or more data processing devices that implement or execute, in conjunction with other devices, such as one or more of those in the system 100, control programs associated with some of the various aspects of the disclosure. Each of the phrases “data processing device,” “data processor,” “processor,” and “computer” is intended to include any data processing device, such as a central processing unit (“CPU”), a circuit, a field programmable gate array (FPGA), a desktop computer, a laptop computer, a mainframe computer, a tablet computer, a personal digital assistant, a cellular phone, and any other device configured to process data, manage data, or handle data, whether implemented with electrical, magnetic, optical, biological components, or the like.

The memory device system 130 includes one or more processor-accessible memory devices configured to store information, including the information needed to execute the control programs associated with some of the various aspects of the disclosure. The memory device system 130 may be a distributed processor-accessible memory device system including multiple processor-accessible memory devices communicatively connected to the data processing device system 110 via a plurality of computers and/or devices. On the other hand, the memory device system 130 need not be a distributed processor-accessible memory system and, consequently, may include one or more processor-accessible memory devices located within a single data processing device.

Each of the phrases “processor-accessible memory” and “processor-accessible memory device” is intended to include any processor-accessible data storage device, whether volatile or nonvolatile, electronic, magnetic, optical, or otherwise, including but not limited to, registers, floppy disks, hard disks, Compact Discs, DVDs, flash memories, ROMs (Read-Only Memory), and RAMs (Random Access Memory). In some aspects of the disclosure, each of the phrases “processor-accessible memory” and “processor-accessible memory device” is intended to include a non-transitory computer-readable storage medium. In some aspects of the disclosure, the memory device system 130 can be considered a non-transitory computer-readable storage medium system.

The phrases “communicatively connected” and “communicatively coupled” are intended to include any type of connection, whether wired or wireless, between devices, data processors, or programs in which data may be communicated. Further, the phrases “communicatively connected” and “communicatively coupled” are intended to include a connection between devices or programs within a single data processor, a connection between devices or programs located in different data processors, and a connection between devices not located in data processors at all. In this regard, although the memory device system 130 is shown separately from the data processing device system 110 and the input-output device system 120, one skilled in the art will appreciate that the memory device system 130 may be located completely or partially within the data processing device system 110 or the input-output device system 120. Further in this regard, although the input-output device system 120 is shown separately from the data processing device system 110 and the memory device system 130, one skilled in the art will appreciate that such system may be located completely or partially within the data processing device system 110 or the memory device system 130, depending upon the contents of the input-output device system 120. Further still, the data processing device system 110, the input-output device system 120, and the memory device system 130 may be located entirely within the same device or housing or may be separately located, but communicatively connected, among different devices or housings. In the case where the data processing device system 110, the input-output device system 120, and the memory device system 130 are located within the same device, the system 100 of FIG. 1 can be implemented by a single application-specific integrated circuit (ASIC) in some aspects of the disclosure.

The input-output device system 120 may include a mouse, a keyboard, a touch screen, another computer, or any device or combination of devices from which a desired selection, desired information, instructions, or any other data is input to the data processing device system 110. The input-output device system 120 may include any suitable interface for receiving information, instructions or any data from other devices and systems described in various ones of the aspects of the disclosure.

The input-output device system 120 also may include an image generating device system, a display device system, a speaker device system, a processor-accessible memory device system, or any device or combination of devices to which information, instructions, or any other data is output from the data processing device system 110. In this regard, if the input-output device system 120 includes a processor-accessible memory device, such memory device may or may not form part or all of the memory device system 130. The input-output device system 120 may include any suitable interface for outputting information, instructions or data to other devices and systems described in various ones of the aspects of the disclosure. In this regard, the input-output device system may include various other devices or systems described in various aspects of the disclosure.

FIG. 2 shows an example of a computing device system 200, according to some aspects of the disclosure. In embodiments, the computing device system 200 includes a processor 270, corresponding to the data processing device system 110 of FIG. 1, in some aspects of the disclosure. The memory 251, input/output (I/O) adapter 256, and non-transitory storage medium 257 may correspond to the memory device system 130 of FIG. 1, according to some aspects of the disclosure. The user interface adapter 254, mouse 258, keyboard 259, display adapter 255, and display 260 may correspond to the input-output device system 120 of FIG. 1, according to some aspects of the disclosure. The computing device system 200 may also include a communication interface 252 that connects to a network 253 for communicating with other computing devices.

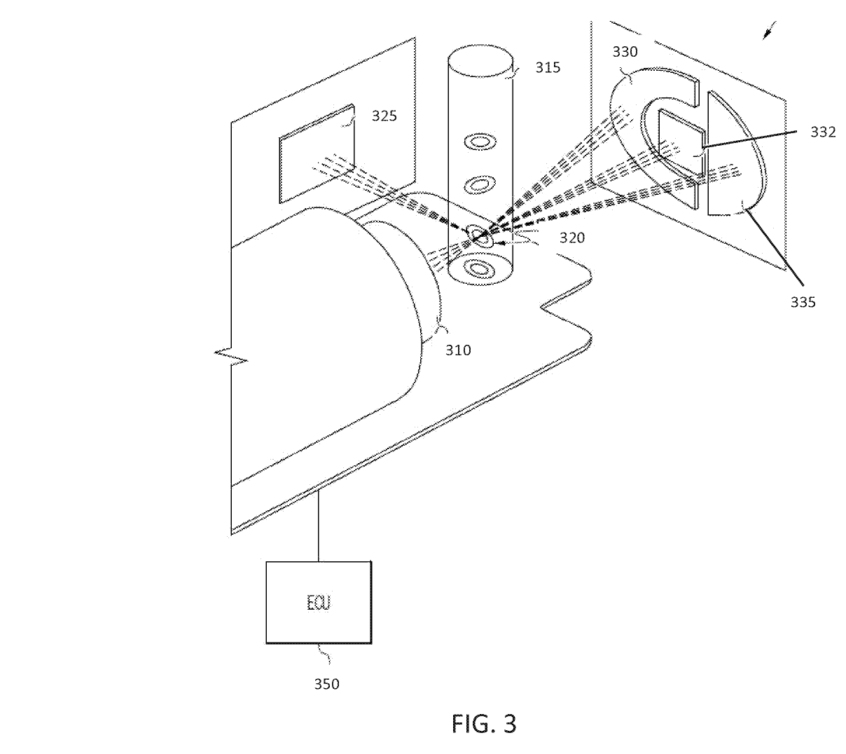

In embodiments, the computing device systems 100, 200 are communicatively coupled to one or more analyzers. For example and referring to FIG. 3, an example analyzer 302 is schematically depicted. In embodiments, the analyzer 302 is a flow cytometry system, which includes an energy source 310, and an electronic control unit (ECU) 350. The analyzer 302, in embodiments, includes a cuvette/flow cell 315 and sensors 325, 330, 332, and 335. The energy source 310 and the sensors 325, 330, 332, and 335 are communicatively coupled to the ECU 350 such that the ECU 350 can send signals to and/or receive signals from the energy source 310 and the sensors 325, 330, 332, 335. The components are illustrated for explanatory purposes and are not drawn to scale.

In operation, as a hematology sample’s constituents 320 (e.g., cells) move one cell at a time through the cuvette/flow cell 315, the energy source 310 emits a beam of energy that is oriented transverse to the axial flow of the sample’s constituents 320 through the cuvette/flow cell 315. The beam of energy emitted by the energy source 310 has a central axis. In embodiments, the beam can be a focused narrow band energy beam (e.g., a LASER) or can be a broadband energy beam.

In examples, a portion of the beam from the energy source 310 that impinges upon the sample’s constituents 320 (e.g., the cells) flowing in the cuvette/flow cell 315 is scattered at a right angle or substantially a right angle to the central axis of the beam of energy (side scattered energy, denoted as “SS”) and is sensed/measured by the SS sensor 325. As used herein, the term “substantially a right angle” means and includes scattered energy which is sensed/measured by SS sensor 325, even though it may not be scattered at exactly a right angle. With respect to energy scattered in the hematology systems described herein, any angle with respect to an axis means and includes such angle in any plane that includes the entire axis, without regard to the direction of the angle (e.g., 3° above an axis and 3° below an axis are both encompassed). As persons skilled in the art will understand, an infinite number of planes wholly include an axis, and an angle as used herein may be in any such plane.

In some examples, varying magnitudes of energy are scattered in all directions from each cell. As such, a magnitude signal may be received from each cell at each sensor angle. Evaluations may be performed of how these responses present together to develop algorithms to classify cells based on their scattering properties. In some examples, the extinction (EXT) sensor 332 may be used to determine absorption as energy is scattered to the various sensors, and a total of energy not transmitted to the EXT sensor 332 may define a magnitude of scattered and absorbed energy.

Another portion of the beam from the energy source 310 that impinges upon the constituents flowing in the cuvette/flow cell 315 is scattered at a much lower angle than 90° with respect to the central axis of the beam of energy. This scatter is termed “low angle forward scattered energy” or “low angle forward scattered low” (FSL) and has an angle range, for example, between approximately 1° to approximately 3° from the central axis of the beam from the energy source 310, inclusive of the endpoints, or can have another angle range that persons skilled in the art will recognize. In the illustrated embodiment, the FSL sensor 335 is oriented to capture/measure the low angle forward scatter energy and is oriented at approximately 1° to approximately 3° from the central axis of the beam of the energy source 310, inclusive of the endpoints.

In the depicted analyzer 302, various other energy may be sensed/measured, and persons skilled in the art will recognize them. In embodiments, such other energy include extinction/axial energy (EXT) (e.g., from approximately 0° to approximately 0.5° from the central axis, inclusive of the endpoints), which is sensed/measured by the EXT sensor 332, and “high angle forward scattered energy” or “forward scattered high” (FSH) (e.g., from approximately 4° to approximately 9° from the central axis, inclusive of the endpoints), which is sensed/measured by the FSH sensor 330. Such energy and angle ranges are exemplary, and other energy and other angle ranges will be recognized by persons skilled in the art. In embodiments, a time metric called time-of-flight (TOF) may be measured and analyzed. As persons skilled in the art will recognize, TOF refers to the amount of time that a sample’s constituent (e.g., a cell) is interrogated by the beam from the energy source 310. TOF may be determined based on EXT energy sensed/measured by EXT sensor 332 or based on readings from the FSL sensor 335. In embodiments, fluorescence energy may be sensed/measured. The disclosure below may refer to one or more of SS, FSL, FSH, EXT, TOF and fluorescence, as examples of energy and metrics that can be used in accordance with aspects of the present disclosure. It is intended and will be understood that other flow cytometry and/or hematology system signals and metrics not expressly mentioned herein are also encompassed within the scope of the present disclosure. Furthermore, the configuration of sensors 325, 330, 332, 335 in FIG. 3 is exemplary. In embodiments, the analyzer 302 may be implemented by other configurations of sensors or components different from those shown in FIG. 3.

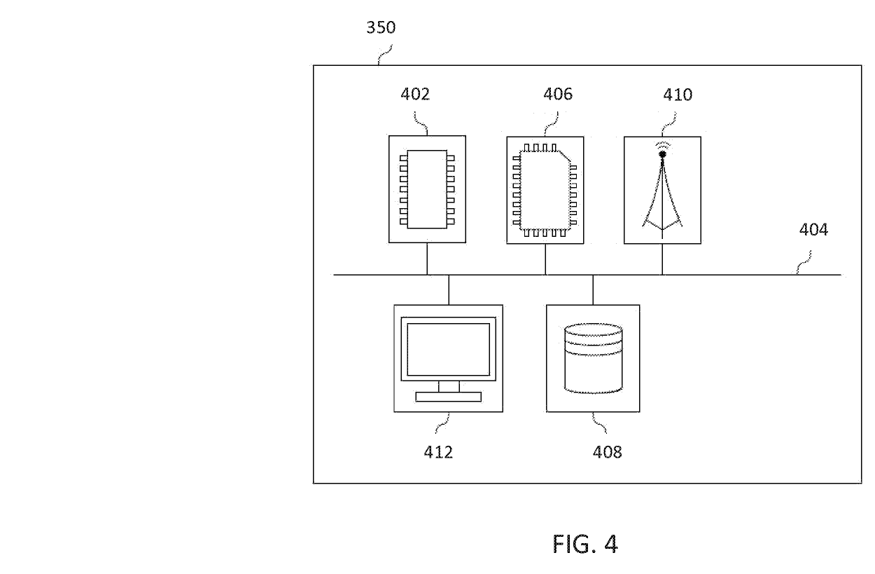

FIG. 4 schematically depicts an example configuration of the ECU 350 of the analyzer 302 of FIG. 3. In the illustrated example, the ECU 350 includes one or more processors 402, a communication path 404, one or more memory modules 406, a data storage component 408, network interface hardware 410, and an output device 412.

Each of the one or more processors 402 may be any device capable of executing machine readable and executable instructions. Accordingly, each of the one or more processors 402 may be a controller, an integrated circuit, a microchip, a computer, or any other physical or cloud-based computing device local to or remote from the analyzer 302 (FIG. 3). The algorithms, including the trained models, signal preprocessing, and noise removal methods discussed below, may be executed by the one or more processors 402. The one or more processors 402 are communicatively coupled to a communication path 404 that provides signal interconnectivity between various modules of the ECU 350. Accordingly, the communication path 404 may communicatively couple any number of processors 402 with one another, and allow the modules coupled to the communication path 404 to operate in a distributed computing environment. Specifically, each of the modules may operate as a node that may send and/or receive data.

The communication path 404 may be formed from any medium that is capable of transmitting a signal such as, for example, conductive wires, conductive traces, optical waveguides, or the like. In some embodiments, the communication path 404 may facilitate the transmission of wireless signals, such as WiFi, Bluetooth®, Near Field Communication (NFC) and the like. Moreover, the communication path 304 may be formed from a combination of mediums capable of transmitting signals. In one embodiment, the communication path 404 comprises a combination of conductive traces, conductive wires, connectors, and buses that cooperate to permit the transmission of electrical data signals to components such as processors, memories, sensors, input devices, output devices, and communication devices. Additionally, it is noted that the term "signal" means a waveform (e.g., electrical, optical, magnetic, mechanical or electromagnetic), such as DC, AC, sinusoidal-wave, triangular-wave, square-wave, vibration, and the like, capable of traveling through a medium.

The ECU 350 includes the one or more memory modules 406 communicatively coupled to the communication path 404. The one or more memory modules 406 may comprise RAM, ROM, flash memories, hard drives, or any device capable of storing machine readable and executable instructions such that the machine readable and executable instructions can be accessed by the one or more processors 402. The machine readable and executable instructions may comprise logic or algorithm(s) written in any programming language of any generation (e.g., 1GL, 2GL, 3GL, 4GL, or 5GL) such as, for example, machine language that may be directly executed by the processor, or assembly language, object-oriented programming (OOP), scripting languages, microcode, etc., that may be compiled or assembled into machine readable and executable instructions and stored on the one or more memory modules 406. Alternatively, the machine readable and executable instructions may be written in a hardware description language (HDL), such as logic implemented via either a field-programmable gate array (FPGA) configuration or an application-specific integrated circuit (ASIC), or their equivalents. Accordingly, the methods described herein may be implemented in any conventional computer programming language, as pre-programmed hardware elements, or as a combination of hardware and software components.

Referring still to FIG. 4, the example ECU 350 includes the data storage component 408. The data storage component 408 may store data captured by the analyzer 102 (FIG. 3), as disclosed in further detail below. The data storage component 408 may also store other data used by the various components of the ECU 350.

In embodiments, the ECU 350 comprises network interface hardware 410 for communicatively coupling the analyzer 102 (FIG. 3) to the system 100 (FIG. 1) or the system 200 (FIG. 2). This may allow data to be shared between the devices to improve the data collected by the devices, as disclosed herein.

In some embodiments, the ECU 350 comprises the output device 412. The output device 412 can include a graphical user interface (GUI), a screen, one or more devices in communication with the one or more processors 402 (such as smartphones, tables, and the like), and/or any other device or interface suitable for displaying data. In some examples, the output device 412, or another device, may be configured to as an input device to receive user input.

While in the example shown in FIGS. 2 and 3, the analyzer 302 is depicted as being a flow cytometer, it should be understood that the one or more analyzers may include any suitable analyzer, including and without limitation, blood chemistry analyzers, urine chemistry analyzers, urine sediment analyzers, rapid assay analyzers, and the like.

Referring to FIGS. 1- 3, and 6-8, various methods 600, 700, and 800 may be performed by way of associated computer-executable instructions according to some example aspects of the disclosure. In various example aspects of the disclosure, a memory device system (e.g., memory device system 130 / memory 251) is communicatively connected to a data processing device system (e.g., data processing device systems 110/ processor 270, otherwise stated herein as “e.g., 110”) and stores a program executable by the data processing device system to cause the data processing device system to execute various aspects of methods 600, 700, and/or 800 via interaction with at least, for example, various databases. In these various aspects of the disclosure, the program may include instructions configured to perform, or cause to be performed, various ones of the instructions associated with execution of various aspects of methods 600, 700, and/or 800. In some aspects of the disclosure, methods 600, 700, and 800 may include a subset of the associated blocks or additional blocks than those shown in FIGS. 6-8. In some aspects of the disclosure, methods 600, 700, and 800 may include a different sequence indicated between various ones of the associated blocks shown in FIGS. 6-8.

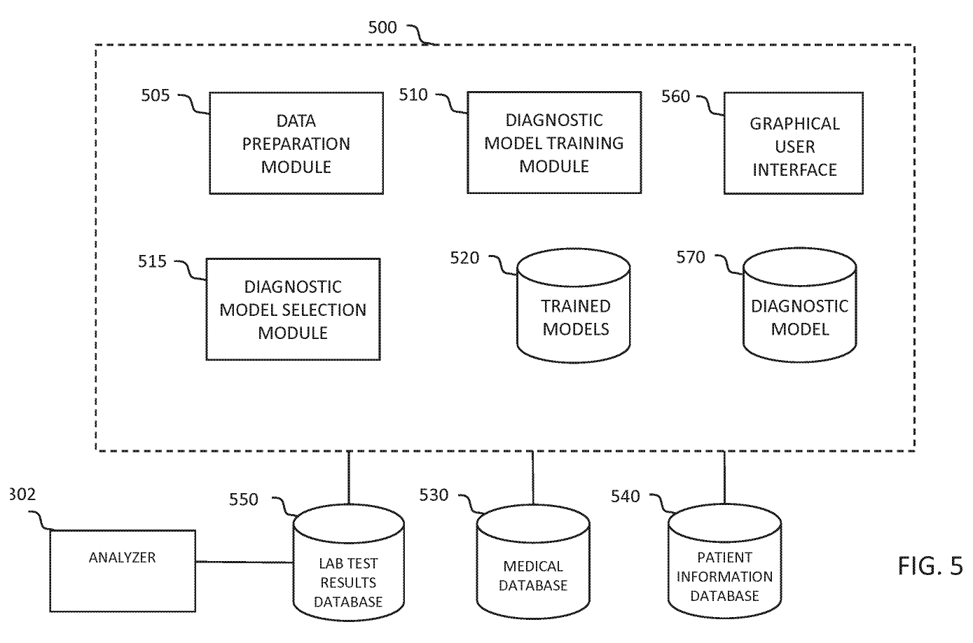

FIG. 5 shows an example of a diagnostic system 500 for assessing the risk of PLN, according to some aspects of the disclosure. The system 500 may be a particular implementation of the systems 100, 200 according to some aspects. In some aspects of the disclosure, the diagnostic system 500 is implemented by programmed instructions stored in one or more memories and executed by one or more processors of the systems 100, 200.

In some aspects of the disclosure, the diagnostic system 500 includes a data preparation module 505, a diagnostic model training module 510, a diagnostic model validation/selection module 515, and one or more diagnostic models 520. In some aspects of the disclosure, the diagnostic system 500 may be communicatively connected to one or more databases 530, 540, and 550. In some aspects of the disclosure, the diagnostic system 500 includes a graphical user interface 560 to permit a user to interact with the system 500. In some aspects of the disclosure, the diagnostic system 500 includes a knowledge based diagnostic model 570 which interacts with the user via the graphical user interface 560 to provide advanced guidance and diagnosis information. In some aspects of the disclosure, the one or more diagnostic models 520 may be stored in a database. For example, in embodiments, the lab test results database 550 contains lab test results from the one or more analyzers 302.

In some aspects of the disclosure, a medical database 530 stores reference medical data such as ranges of normal, low, and high test results for various diagnostic tests performed in the veterinary clinic or at a veterinary reference laboratory using various diagnostic testing instruments. In some aspects of the disclosure, the diagnostic tests may be performed by mobile laboratories, using home testing kits, etc. In some aspects of the disclosure, the diagnostic system 500 may access the medical database 530 to compare actual patient test results, stored in a laboratory test results database 550, with the typical ranges stored in the medical database 530 to interpret the test results performed at the veterinary clinic, the veterinary reference laboratories or using other means. In some aspects of the disclosure, the diagnostic tests include complete blood count (CBC), blood chemistry, PCR assays, etc. performed on the one or more analyzers 302.

In some aspects of the disclosure, a patient information database 540 stores a patient’s medical record, including patient demographic information, vital signs at each clinical visit, diagnoses, medications, treatment plans, progress notes, patient problems, vaccine history, test results, and imaging data such as radiographs. The demographic data may include species, breed, weight, age, gender or sex, and geographic location, for example. In some aspects of the disclosure, the medical record may also include information on test results (for example, CBC, blood chemistry, pathology, urinalysis, serology, and PCR (polymerase chain reaction) panels/assays), vector of exposure, and diagnoses, obtained from the laboratory test results database 550.

In some aspects of the disclosure, the diagnostic system 500 is deployed on a point of care (POC) terminal located at a veterinarian’s office or a clinic. The POC terminal may be connected to the veterinary reference laboratories or the diagnostic testing instruments at the veterinary clinic to receive test results for a patient. In some aspects of the disclosure, the POC terminal may be connected to one or more software servers. In some aspects of the disclosure, the software servers provide centralized software development resources and support for generating machine learning models (diagnostic models 520 and 570) for assessing the risk of PLN. In some aspects of the disclosure, the diagnostic models 520 and 570 may be deployed and executed locally on the POC terminal. In other some aspects of the disclosure, the diagnostic system 500 may be deployed on the server, with the POC terminal acting as a “client” that connects to the server, which executes the diagnostic models 520 and 570 based on patient information transmitted to the server from the POC terminal.

In some aspects of the disclosure, the diagnostic models 520 are used to provide alerts to clinicians when a patient shows an increased risk of PLN. In some aspects of the disclosure, further targeted screening may be performed using diagnostic model 570, in response to the alert, to validate the presence/absence of PLN. This approach provides significant advantages for recognizing and treating PLN by reducing the number of missed diagnoses of early-stage PLN prior to advanced disease, while reducing unnecessary diagnostic and treatment costs.

Generating machine learning models to assess the risk of PLN poses several challenges. Conventional machine learning models work best when there is a large amount of training data with a relatively uniform distribution of positive and negative examples available, and the patterns that suggest the presence (positive) or absence (negative) of a particular disease are well-defined and separable from each other in the feature space. While PLN is relatively common in animals such as cats and dogs, confirmed diagnoses are underrepresented in medical records. For example and as noted above, PLN may exist in as many as 50% of dogs. However, because urinalysis is typically performed in only 10-20% of veterinary visits in the United States, PLN is typically not timely and/or accurately diagnosed. Moreover and as discussed in further detail below, veterinary medical records do not typically indicate a confirmed diagnoses, even when a veterinarian may have made an affirmative diagnoses of PLN. Thus, the training set for the diagnostic model for PLN is highly skewed, with much fewer examples of positive data samples than negative data samples. Moreover, the patterns (in feature space) for positive data samples (from patients with PLN) are not well-differentiated and vary greatly, which makes it difficult to separate the clusters of positive samples from the clusters of negative samples (intermingling of positive and negative samples in the feature space) when training the diagnostic models 520 and 570.

To address these difficulties, a multi-stage approach is used to generate the diagnostic models 520 in some aspects of the disclosure. In some aspects of the disclosure, the diagnostic models 520 for assessing the risk for PLN are generated using supervised machine learning method. The supervised method involves utilizing very large sets of anonymized patient data to provide a labeled training set of defined positive cases (patients who have the attribute of interest) and negative cases (patients without the attribute of interest). In some aspects of the disclosure, criteria for inclusion or exclusion of data samples in the training data set, and the definitions of positive and negative data samples, are defined by human medical experts. The computing device system 200, when presented with the training set, looks backwards at patient data samples in the months prior to the diagnosis of PLN. From this data, the computing device system 200 uses mathematical algorithms to learn the patterns and relationships of weighted analytes and trends, along with other patient data, such as signalment, until one or more diagnostic models 520 that best predict the eventual diagnosis of PLN are generated. Once the diagnostic models 520 are generated, they go through a preclinical technical validation for performance requirements on labeled data followed by a clinical evaluation in the field on unlabeled patient results before deployment.

In some aspects of the disclosure, a training data set with a wide patient population showing diverse clinical presentations is collected. This permits the diagnostic models 520 to encode a more realistic representation of positive cases seen in general practice. The collected data set is filtered to remove data samples that represent outliers that could improperly skew the diagnostic models 520. For example, data samples from patients who are already engaged in a treatment protocol for PLN are removed from the training set, to ensure that the training set represents “natural” untreated presentations of PLN. Similarly, data samples obtained from patients after a positive diagnosis for PLN, or after a urinalysis or other tests, are removed because the patent may have been administered treatment that masks the natural indicators of PLN after positive diagnosis or based on the result of these tests.

In some aspects of the disclosure, the data preparation module 505 communicates with the one or more databases 530, 540, 550 to receive laboratory test data, and medical record and medical history data for a large population of patients, gathered over a period of time. In some aspects of the disclosure, the medical record includes biographical information, such as age, gender or sex, breed, and geographic location, one or more of which may be used as input features in training the machine learning models. These features can provide contextual discriminatory power to the machine learning models. In addition to biographical information, the medical record and the medical history data include information on any diagnostic tests that have been performed, and patient notes entered by a veterinarian. For example, a complete blood count (CBC) test includes data (results) for red blood cell count, hematocrit, hemoglobin, mean cell volume, mean corpuscular hemoglobin, mean corpuscular hemoglobin concentration, red blood cell redistribution width, reticulocytes (percentage and number), reticulocyte hemoglobin, nucleated red blood cells, white blood cell count, neutrophils (percentage and number), lymphocytes (percentage and number), monocytes (percentage and number), eosinophils (percentage and number), basophils (percentage and number), band neutrophils, platelet count, platelet distribution width, mean platelet volume, plateletcrit, total nucleated cell count, agranulocytes (percentage and number), and granulocytes (percentage and number). Other tests may check for presence, count, or concentration of squamous epithelial cells, non squamous epithelial cells, bacteria (rods or cocci), hyaline and nonhyaline casts, or crystals (bilirubin, ammonium biurate, struvite etc.). Levels of various proteins, enzymes, or minerals may have been checked. Other tests to check for specific pathogens may have been run, and their results stored in the medical record and the medical history data. Information on the diagnosis of PLN, and the date of the diagnosis may also be stored in the medical record and medical history data.

In some aspects of the disclosure, the data preparation module 505 receives the laboratory test data and the medical record and medical history data, and extracts features to be used as inputs for training machine learning models to predict the risk of PLN. Ground truth (labeling of the data samples as positive or negative examples of PLN) may be obtained from the diagnosis information, if expressly stored in the medical record and medical history data. For example, in some aspects of the disclosure, positive examples may be defined as data samples from patients with a clinical diagnosis of PLN. In some aspects of the disclosure, positive examples may be defined as data samples from patients that have been prescribed treatment protocols consistent with the treatment of PLN, regardless of whether the medical record contains an affirmative clinical diagnosis of PLN. For example and without being bound by theory, in the human health space, health records often include insurance-based diagnostic codes indicating conclusive diagnoses. While done for the purpose of categorizing insurance payments, these insurance-based diagnostic codes additionally provide documentation of clinical diagnoses in human medical records.

Because of the relative lack of insurance in the veterinary space, veterinary health records do not generally include insurance-based diagnostic codes. Consequently, veterinary health records typically not include conclusive diagnoses, frustrating the ability to identify ground truth for machine learning purposes. Accordingly, in embodiments, identification of positive samples may be based on data samples that include prescribed treatment protocols consistent with the treatment of PLN in conjunction with clinical signs and/or lab results consistent with the presentation of PLN.

Treatment protocols for PLN may include the prescription of angiotensin converting enzyme (ACE) inhibitors, such as enalapril or benazepril, asprin to prevent clotting, beta-blockers, calcium channel inhibitors. In some circumstances, diet changes to address food allergies can be prescribed, and/or anti-parasitics may be administered.

The remaining data samples, which do not meet the criteria for positive data samples, are labeled as negative examples. The data preparation module 505 also filters the collected data samples based on predefined exclusion criteria to generate a labeled training dataset.

In some aspects of the disclosure, the data preparation module 505 determines the ratio of positive and negative labeled data samples in the training dataset and uses data sampling techniques to achieve a more balanced training dataset. For example, if the training data set has 10 times as many negative labeled examples as positive labeled examples, the data preparation module 505 may sample the negative examples to select 1 out of every 5 negative examples, to achieve a dataset that has twice as many negative labeled data samples as positive labeled data samples. It is readily understood that any predefined ratio for negatively labeled data samples to positively labeled data samples may be used, and the above example is provided merely for purposes of illustration only.

Data sampling techniques include random sampling, stratified sampling, cluster sampling, importance sampling, uncertainty sampling, and active learning, as examples. Random sampling entails randomly selecting the desired number of data samples from the larger pool of data samples. Random sampling is simple and cost effective, generally ensures that the selected subset is a representative sample of the overall data distribution and can be implemented by randomly selecting a fixed number of instances or by specifying a percentage of the collected data samples to be included in the training set. Stratified sampling is useful when the class distribution of the selected data samples needs to be maintained. For example, in a case where the larger pool of collected data samples (negative examples) can be divided into different classes or categories, each class or category is proportionally represented in the selected data samples to ensure diversity of the data. Cluster sampling involves partitioning the data into clusters based on certain criteria (e.g., geographic location, demographics, or other relevant factors). Instead of sampling individual instances of patient data samples, each cluster is samples to ensure that a representative data set is collected. This can be beneficial when clusters represent distinct groups or subpopulations within the data, such as different breeds of animals. Importance sampling assigns weights to each data sample based on its importance or relevance to the diagnostic task. Data samples that are more informative or challenging can be given higher weights, while less informative samples receive lower weights. This technique allows prioritizing the inclusion of important data samples in the training dataset. Uncertainty sampling focuses on selecting data samples that the current model is uncertain about or finds challenging to classify. By selecting these data samples, the diagnostic system actively targets areas of the data where the trained machine learning model needs further improvement. Common uncertainty sampling strategies include selecting instances with the highest prediction entropy, margin, or confidence scores. Active learning is an iterative sampling approach where the trained machine learning model interacts with the data preparation module 505 to select the most informative data samples for further training. The model can query the diagnostic system for instances it is uncertain about or instances that are expected to have the most impact on improving its performance. This interactive process helps optimize the data selection based on the evolving needs of the machine learning models.

In some aspects of the disclosure, the data samples in the training set are represented using feature vectors that include age, species, breed, sex, and various analytes such as electrolyte levels (sodium, potassium, chloride, sodium-to-potassium ratio), glucose, urea (blood urea nitrogen - BUN), red blood cell count (RBC), monocytes, lymphocytes, & lymphocytes reticulocyte, %reticulocyte, neutrophils, %neutrophils, eosinophils, %eosinophils, cholesterol, amylase, and calcium. It should be understood that the features included in the data samples are not limited to those listed here, and this list is provided for purposes of illustration only. Other features could be obtained from the patient’s medical record and medical history or various laboratory tests such as CBC, basic chemistry panels, or more extensive chemistry panels, etc.

The feature space for assessing the risk of PLN is very complex because of the potential number of features and because not all patients will have available information for all features. Complex feature spaces pose challenges for machine learning for several reasons such as dimensionality, computational complexity, overfitting, sparsity etc. As the number of potential features (dimensions) that can be used for assessing the risk of PLN increases, the volume of the feature space grows exponentially. In high-dimensional spaces, the available data becomes sparse, making it challenging for machine learning algorithms to generalize well. Models may struggle to find patterns or relationships when the ratio between the number of features and the number of samples increases.

With an increasing number of features, the computational cost of training and evaluating machine learning models tends to rise. Many algorithms have time and space complexity that scales with the number of dimensions, making them computationally expensive in high-dimensional spaces. High-dimensional spaces also increase the risk of overfitting, where models capture noise or specific characteristics of the training data that do not generalize well to new, unseen data. This is also a direct consequence of the sparsity of data in complex feature spaces. Models may fit the training data too closely, capturing spurious correlations or outliers, rather than learning the underlying patterns, because the available data points may be sparsely distributed. This sparsity makes it difficult for machine learning models to identify meaningful relationships between features and the target variable. Insufficient (sparse) data can also lead to poor model performance and unreliable predictions.

Some machine learning algorithms, especially those with a large number of tunable parameters, may become more difficult to train and fine-tune in high-dimensional spaces. This can lead to challenges in finding the right machine learning models for use in assessing the risk of PLN. Moreover, some algorithms may become numerically unstable in high-dimensional spaces, leading to issues such as a lack of convergence during training or difficulties in optimizing the model parameters.

In some aspects of the disclosure, the complexity of the feature space is reduced by performing feature selection – identifying only the most relevant/discriminative features and removing other features that do not significantly boost the performance of the machine learning models. However, it can be difficult to distinguish between informative and irrelevant features, leading to suboptimal model performance. To address these challenges, feature engineering, dimensionality reduction techniques (e.g., principal component analysis), and careful model selection are crucial.

In some aspects of the disclosure, the diagnostic models 520 are used to identify the top predictive features of PLN. To identify features that are most optimal at predicting PLN, each of the diagnostic models 520 is fed data with a similar series of manipulations that are designed to stress the predictive methods of the diagnostic models. For example, in some aspects of the disclosure, the diagnostic models 520 are fed data that omits the patient values for age and are assessed on their ability to predict PLN better than a “random guess”. Each predictive feature is assessed on the proportion of training/validation Monte Carlo analyses that include the respective marker based on being better than the “random guess”.

In some aspects of the disclosure, the top predictive features of PLN include gender or sex, species, breed, CBC, and blood chemistry.

In some aspects of the disclosure, receiver operator characteristics (ROC) curve analyses are performed with machine learning algorithms using the most predictive features for PLN. In some aspects of the disclosure, the diagnostic models 520 are trained on 75% of the data stratified across patient, clinic, and diagnosis, and then validated on the remaining 25% of the data. The ground truth value for a given patient is determined by the clinical diagnosis. The machine algorithms are designed to primarily optimize the area under the curve (AUC) for the ROC curve. This ensures the diagnostic model 520 will be as sensitive (least false negatives) as possible while also being as specific (least false positives) as possible, by striking the right balance between the two. In some aspects of the disclosure, a Monte Carlo analysis was performed on a predetermined number, for example 500, of random training and validation sets to understand the uncertainty of the AUC estimates. In some aspects of the disclosure, a 95% confidence interval estimate for the AUC corresponds to roughly a 65% sensitivity and 88% specificity. It would be obvious to one of ordinary skill in the art that different confidence intervals, corresponding to different sensitivity and specificity rates.

Ensemble learning, which combines multiple models, can be effective in mitigating the impact of complex feature spaces by leveraging the strengths of different models. In some aspects of the disclosure, ensemble learning is used to generate a plurality of machine learning diagnostic models 520 (ensemble of models 520), which are trained using the same or different sets of features. This approach permits the diagnostic system 500 to optimize different diagnostic models 520 on different portions of the sample space, and is especially useful for assessing the risk of PLN due to its varied presentations. The outputs from the various machine learning diagnostic models 520 are merged or combined using ensemble methods to produce a single model/output that encompasses the strengths and compensates for the weaknesses of each individual diagnostic model 520.

In some aspects of the disclosure, the machine learning model is generated by performing data collection, data splitting, model selection, model training, and model validation. Data collection has been discussed in detail above and entails gathering a representative data set that is relevant to assessing the risk of PLN. Since bias and inconsistencies within the data can significantly impact a diagnostic model’s performance, thorough data cleaning and pre-processing are performed. This includes addressing missing values in the data samples, identifying and correcting inconsistencies, and transforming the data into a format suitable for the selected type of machine learning model. In some aspects of the disclosure, feature selection is performed to identify the most discriminative features and, potentially, reduce the complexity of the machine learning model. Once the data set is ready, it is divided into at least two separate training and test sets. In some cases, a separate validation set may also be generated. The training set serves as the labeled ground truth for training the diagnostic models 520 to learn the underlying patterns and relationships within the data. The test set is not presented to the models during training and is used to assess the models’ ability to generalize and predict accurately on new data. The validation set is used during training to help the models generalize and avoid overfitting. During training, the machine learning algorithm iteratively adjusts the internal parameters of each diagnostic model 520 to minimize prediction errors on the training set. This process, known as optimization, aims to ensure that the diagnostic models 520 learn the optimal representations and decision boundaries for accurately classifying the data samples. The choice of appropriate hyperparameters, which control the learning process, plays a crucial role in achieving optimal performance. Hyperparameters govern the learning process and significantly impact the diagnostic model's performance. Tuning these parameters involves finding the optimal settings that maximize model accuracy and generalization. In some aspects of the disclosure, techniques like grid search and random search are employed to systematically explore the parameter space and identify the optimal configuration. Once the diagnostic models 520 are trained, their performance is evaluated using the test set. This involves measuring the models’ accuracy, precision, recall, and other relevant metrics. These metrics provide valuable insights into the models' strengths and weaknesses, allowing for further refinement and improvement. Cross-validation, which involves evaluating the models on multiple splits of the data, provides a more robust estimate of their generalization ability. Both hyperparameter tuning and cross validation can be performed during either of the training or the testing phases, to improve the models’ performance.

Ensemble learning is an advanced machine learning technique that combines predictions from multiple diagnostic models 520 to produce a more accurate and robust final prediction. This approach leverages the diversity of individual diagnostic models 520 to mitigate biases and further improve overall performance, making it particularly effective in complex and varied datasets. The idea behind ensemble learning is that by combining diverse independent models, the weaknesses of one model can be compensated for by the strengths of others, and the aggregation of the models result in a more accurate and reliable prediction. Diversity means that the different machine learning models within the ensemble make different types of errors. This diversity ensures that the ensemble of models 520 can collectively address a broader range of scenarios. The models within the ensemble are designed to be as independent as possible to reduce the risk of systematic errors, because similar models might make the same mistakes. The predictions of individual models 520 are combined through a predefined aggregation strategy, such as averaging, voting, or weighted voting. The goal is to leverage the collective intelligence of the ensemble.

The machine learning models in the ensemble of models 520 can be homogeneous, heterogeneous, or a mix of both. Homogeneous models refer to using more than one machine learning model of the same type, but with intentional variations. For instance, in the case of a decision tree machine learning model, different decision trees having different depths, minimum samples per leaf, or feature subsets maybe generated during training. Heterogeneous models involve combining different types of models, each contributing unique perspectives. This could include combining decision trees with support vector machines, k-nearest neighbors, or neural networks, creating a diverse set of predictors.

In some aspects of the disclosure, one or more ensemble learning training methods are used to train the ensemble of diagnostic models 520. The ensemble learning training methods include bagging, boosting, and stacking. Bagging, also known as bootstrap aggregating, involves generating multiple instances of a same type of machine learning model on different subsets of the training data. Each subset is obtained through bootstrapping (random sampling with replacement or another data sampling technique discussed above). Each instance of the machine learning model is trained independently using one subset of the training data. This process introduces diversity by exposing each model to slightly different instances and reduces overfitting. Different operators, such as the average (for regression) or majority vote (for classification) of the individual model predictions, are used for the final prediction. In some aspects of the disclosure,

Boosting entails building a sequence of diagnostic models, where each subsequent diagnostic model in the sequence tries to reduce the errors of the preceding diagnostic model in the sequence. Boosting assigns weights to instances based on their performance in earlier iterations, emphasizing the importance of misclassified data points. Higher weights are assigned to misclassified instances and lower weights to correctly classified ones and a weak learner is trained on the weighted dataset. The model's performance is evaluated using the test data set and the errors are used to adjust weights. This process is repeated iteratively, giving more emphasis to misclassified instances in each iteration, until the error in prediction is below a desired threshold. Algorithms like AdaBoost and Gradient Boosting exemplify this approach, refining the model iteratively to enhance overall predictive performance.

Stacking involves training multiple different types of machine learning models using the techniques discussed above, and then combining their predictions using another model called a meta-model or blender. The meta-model learns to weigh the predictions of the individual models to create a final prediction.

To ensure robustness and generalization of the trained diagnostic models 520 in the ensemble, both technical (cross-fold) and real-world (clinical trial) validation is employed. This rigorous validation process helps prevent overfitting and provides a more accurate estimate of the models' performance. The predictions from each of the plurality of diagnostic models in the ensemble maybe combined using different functions to generate a single final prediction. For example, in hard voting, the final prediction is determined by the majority vote of the diagnostic models 520. Each diagnostic model has equal weight in the decision-making process. As another example, soft voting considers the confidence or probability assigned to each class by each diagnostic model 520. The final prediction is a weighted combination of these probabilities, providing a more nuanced decision. As yet another example, weighted averaging may be used to assign a weight to each model's prediction based on the model’s performance during validation (technical and/or clinical trial). In weighted averaging, diagnostic models with higher accuracy or reliability are given more significant influence in the final prediction, allowing for a dynamic weighting scheme.

FIGS. 5 and 6 show a flowchart of an ensemble learning based PLN risk assessment method 600, according to various aspects of the disclosure. In step 610, the data preparation module 505 prepares the data sets for training, testing, and validating the diagnostic models 520 for the diagnostic system 500. The raw data samples are obtained from diagnostic tests performed either at laboratories or using diagnostic instruments at the POC terminal, and clinical history data derived from integrated veterinary clinic practice information management software (PIMS). In some aspects of the disclosure, the data samples are collected over a period of time and stored in the one or more databases 530, 540, and 550, discussed above. Ground truth for the data samples may be defined as positive diagnosis of PLN or treatment protocol consistent with a positive diagnosis of PLN. A wide patient population with diverse clinical presentations is included in the training data set to provide the diagnostic models 520 with a more realistic representation of the general patient population. Since the goal of the diagnostic system 500 is to detect early or preclinical cases in addition to more classic presentations, the positively labeled data samples are not restricted to those with clinical signs or to patients in whom PLN is already suspected. In some aspects of the disclosure, medical experts may be used to define a PLN phenotype to label positive data samples (i.e., patients with PLN) and negative data samples (i.e., patients without PLN). The phenotype involves several inclusion and exclusion criteria to label data samples as positive or negative. In some aspects of the disclosure, the collected data samples are divided into a plurality of data subsets for training, validating, and testing the diagnostic models.

The data samples in the training, validating, and testing data sets include features such as cholesterol concentration, age, complete blood count (e.g., mean corpuscular volume (MCV), hemoglobin levels etc.), and serum chemistries (e.g., blood urea nitrogen [BUN], sodium, and potassium) as well as demographic data like gender or sex, breed, and age. The minimum requirements for the diagnostic models include commonly assessed parameters as found in CBC and basic chemistry panels with electrolytes. The model parameters are optimized to meet predetermined performance criteria, for example, specificity of ≥ 95% with sensitivity of at least 70%.

In step 620, the data set is filtered by one or more criteria discussed above to extract the relevant set of data samples for training, validating, and testing the machine learning models 520.

In step 630, the diagnostic model training module 510 selects and trains an ensemble of diagnostic models 520 using the ensemble learning methods discussed previously. In some aspects of the disclosure, in step 630, a plurality of decision trees (random forest of trees) is trained on different feature subsets in the data samples to generate the ensemble of diagnostic models 520. A decision tree is a machine learning model with a tree-like structure where each internal node represents a feature (or attribute) of the data set, and each branch represents a possible outcome of the feature. At each node, the algorithm selects the best feature based on a splitting criterion, such as information gain or Gini impurity. This process continues recursively until reaching a leaf node, which represents the final predicted class or value. Decision trees are easy to interpret and understand, and can be generated using expert domain knowledge (a rule based approach). The tree structure provides a clear visual representation of the decision-making process. Moreover, decisions trees are robust to irrelevant features because the algorithm automatically ignores features that are not relevant for prediction. However, decision trees are prone to overfitting and can become too complex in a large feature space, leading to poor performance on unseen data. Decision trees are also sensitive to noise in the data; slight changes in the data can lead to significant changes in the tree structure. Each tree in a random forest is trained independently and makes its own prediction. The final prediction is then obtained by taking the majority vote from all trees. This approach is more robust to outliers and noise in the data.

Random forests are ensemble learning algorithms that combine multiple decision trees to improve overall performance. They work by building a forest of individual decision trees, each trained on a different subset of the data and using a random subset of features at each split. The predictions from these individual trees are then combined to make the final prediction. Random forests provide improved accuracy and robustness over individual decision trees. By combining multiple decision trees, random forests are less prone to overfitting and more robust to noise in the data, and can handle complex relationships between features. Random forests can learn complex relationships between features that may not be easily captured by a single decision tree. Although more computationally expensive to train, and less interpretable than individual decision trees, the random forest ensemble models outperform individual decision trees because assessing the risk of PLN is a complex problem with many features and intricate relationships.

In some aspects of the disclosure, in step 630, a gradient boosting approach is used to build a sequence of decision trees using ensemble learning. Gradient boosting is a powerful technique for building ensemble models by sequentially adding weak machine learning models (models with low individual accuracy) to improve overall performance. The training process starts with an initial model, often a simple model like a single decision tree. The algorithm calculates the pseudo-residuals, which represent the difference between the actual and predicted values for each data sample, and iteratively adds weak machine learning models. In each iteration, a new weak machine learning model is trained on the current pseudo-residuals. This new machine learning model focuses on correcting the errors made by the previous machine learning models. The learning rate determines the weight given to each new machine learning model in the ensemble. The (weighted) predictions of all weak machine learning models are combined to generate the final prediction at each iteration. The iteratively process of adding weak machine learning models and updating predictions continues until a stopping criterion is met. Common stopping criteria include reaching a desired level of accuracy or exceeding a maximum number of iterations. Some benefits of using gradient boosting to build an ensemble model include improved accuracy, reduced variance, flexibility, and scalability. By combining the predictions of multiple weak machine learning models, gradient boosting can achieve significantly higher accuracy than individual learners. Predictions are made by majority vote of the weak machine learning models; predictions, weighted by their individual accuracy. Further, since gradient boosting focuses on correcting errors made by previous machine learning models, leading to a more robust ensemble model with lower variance. Gradient boosting can be used with various types of machine learning models, such as decision trees, linear regression models, and others, making it easy to generate heterogenous models for the ensemble of diagnostic models.

In steps 640 and 650, the diagnostic model selection module 515 performs technical and clinical validation of the trained ensemble of diagnostic models 520, respectively. Technical validation, performed in step 640, ensures that the diagnostic models 520 meet the performance requirements, are unbiased, and are strong enough to support a clinical evaluation prior to deployment. The preclinical technical validation is performed by running the model against the validation and test data sets of cases. In some aspects of the disclosure, the training set is used to train the ensemble model, the validation set is used to tune hyperparameters of the ensemble model and monitor the model for overfitting during training. The test set is used to evaluate the final model's performance on unseen data. The machine learning models used in ensemble learning (whether random forest, gradient boosting models, or other machine learning models) have several hyperparameters that can significantly impact their performance. Techniques like grid search or random search can be used to identify the optimal values of these hyperparameters. The validation data set is essential for evaluating the performance of different hyperparameter configurations and selecting the best ones for generating the structure of the machine learning models included in the ensemble. In some aspects of the disclosure, metrics such as loss function, accuracy, and AUC can be monitored during model training and validation to improve the model’s performance.

In some aspects of the disclosure, a loss function measures the difference between the predicted and actual values for a data sample. Lower values of the loss function indicate better model performance. Common loss functions include mean squared error (MSE), which measures the average squared difference between predicted and actual values, cross-entropy, which measures the difference between the predicted probability and the actual class, and logarithmic loss, which is like cross-entropy but more sensitive to misclassified examples. Accuracy represents the percentage of predictions that are correct. It is a simple and intuitive metric but can be misleading when dealing with imbalanced datasets. AUC (area under the curve) measures the ability of a classifier to distinguish between classes. The AUC represents the area under the receiver operating characteristic (ROC) curve, which plots the true positive rate (TPR) against the false positive rate (FPR) at different thresholds. An AUC of 1 represents a perfect classifier, while an AUC of 0.5 represents a random guess. In some aspects of the disclosure, one or more of the loss function, accuracy, and AUC are observed. If these metrics plateau or worsen on the validation data set, the trained ensemble model likely suffers from overfitting. In this case, early stopping techniques are used to stop training once the validation metrics start declining, preventing overfitting.

In some aspects of the disclosure, cross validation is used to monitor the ensemble diagnostic model 520 for overfitting. Cross-validation is a resampling procedure used to evaluate machine learning models on a limited data sample. The procedure has a single parameter called k that refers to the number of groups that a given data set is to be split into. As such, the procedure is often called k-fold cross-validation. When a specific value for k is chosen, such as k=10, the procedure becomes 10-fold cross-validation. Cross-validation is primarily used to estimate how the trained model is expected to perform in general when used to make predictions on data not used during the training of the models 520. The dataset is shuffled randomly and divided into a predefined number (k) of groups. The training and validation process is performed k times, with one of the groups of data being held out as the validation set for each iteration and the remaining k-1 groups being used as the training set. Each model is fitted (trained) on the training set and evaluated (validated) on the test set to determine the level of generalization of the trained models. The purpose of k-fold cross validation is not to pick one of the trained models 520 as the final machine learning model but, rather, to help determine the model structure and the hyperparameter tuning process for each machine learning model 520. Once the ensemble model 520 is trained and hyperparameters are tuned, its performance is evaluated using the test data set.

In step 650, a clinical validation of the ensemble of diagnostic models 520 is performed to measure the model’s effectiveness in assessing the risk for PLN, prior to deployment (step s660). In some aspects of the disclosure, the clinical validation is performed by deploying the diagnostic models 520 on a test basis at a selected number of clinics, evaluating new data samples from patients using the trained diagnostic models 520, and validating the prediction of the diagnostic models 520 using expert opinion of the veterinarians. Further diagnostic tests may be run, in the case where the ensemble model 520 predicts a higher risk of PLN, to determine whether the patient is suffering from PLN.

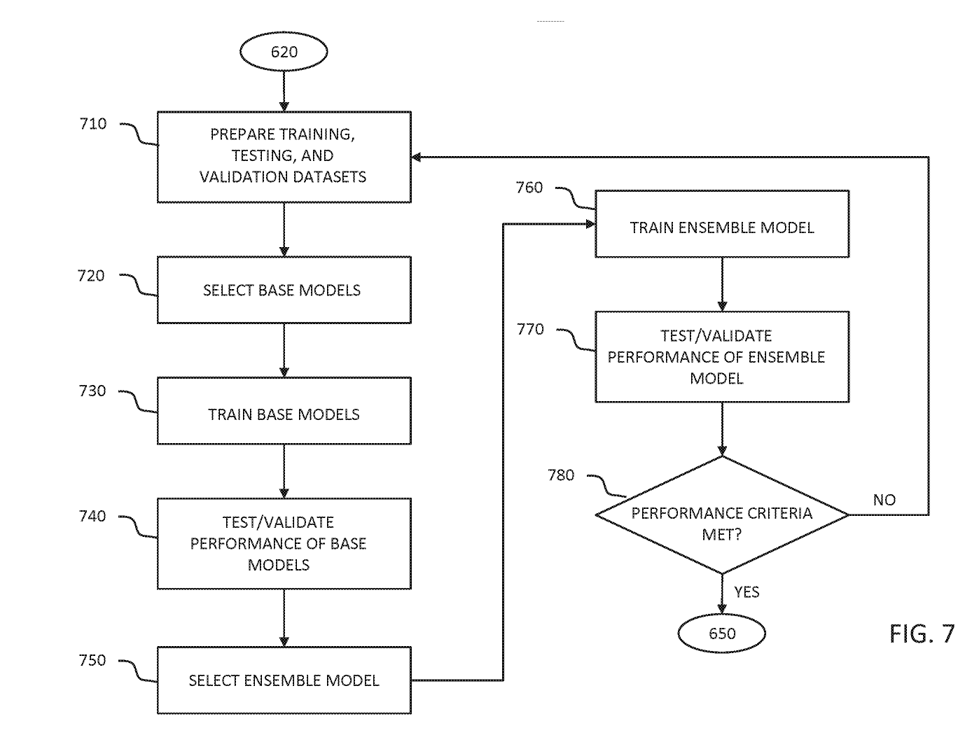

FIGS. 5 and 7 show a flowchart with additional details for a method 700 of training the ensemble of diagnostic models 520, according to some aspects of the disclosure. In step 710, the collected and filtered medical data (from steps 610 and 620 of FIG. 6) is pre-processed and split into training, testing, and validation datasets using the sampling techniques discussed above.

In step 720, the individual base models (learners) for the ensemble of models are selected. The learners may include one or more machine learning models such as decision trees, random forests, support vector machines, neural networks etc. A diverse set of learners may be selected for better ensemble performance. Multiple instances of a same type of machine learning model with different architecture or parameter sets may also be selected.

In step 730, each base model (learner) is trained independently on the training data set using model-specific training methods.

In step 740, each base model is tested/validated for performance using technical validation methods such as cross-validation. The training and validating steps 730 and 740 are repeated iteratively until predefined performance criteria for each base model is met. Performance metrics (e.g., accuracy, loss) on training and testing/validation data are measured and tracked to improve the performance of each base model. In some aspects of the disclosure, early stopping is used to prevent overfitting during the training step 730 based on the performance metrics. During the training and validating steps 730 and 740, the hyperparameters (e.g., tree depth, number of estimators etc.) for each model are tuned.

In step 750, the ensemble architecture (e.g., bagging, boosting, stacking), the learning approach (e.g., parallel, sequential), and the prediction aggregation strategy (e.g. averaging, voting) is selected.

In step 760, the ensemble of diagnostic models 520 is trained using the outputs or predictions of the base models. In some aspects of the disclosure, the base models may be weighted differently based on their performance, permitting models with better performance to have more influence on the output of the ensemble model 520.

In step 770, the ensemble model's performance is validated on unseen validation data. The ensemble model’s performance is also compared with individual base model performance to check for overfitting (high training accuracy, low validation accuracy). In some aspects of the disclosure, in step 780, results from the ensemble model 520 are analyzed. If the performance meets the desired criteria, the ensemble model is ready for clinical validation and deployment. If the performance does not meet the desired criteria, areas for improvement are identified and steps 710- 770 are repeated to adjust the base models, ensemble architecture, hyperparameters, or data pre-processing as needed. Training and validation of the individual learners and the ensemble model are repeated until the desired performance is achieved.

In some aspects of the disclosure, the diagnostic system 500 includes a graphical user interface 560 that permits a user to further engage with an interactive diagnostic model 570 when any of the diagnostic models in the ensemble of models 520 indicate a risk of PLN. In these aspects of the disclosure, the ensemble of diagnostic models 520, discussed above, provides an indication that the veterinarian should perform additional screening to assess the risk for PLN.

In some aspects of the disclosure, the additional screening is performed in an interactive manner by a user (for example, the veterinarian) engaging with the interactive diagnostic model 570 via the user interface 560. The user interface presents a sequence of questions, dynamically selected by the third diagnostic model 570 based on answers provided to previous questions, to guide the veterinarian towards additional testing to confirm the presence or absence of PLN.

In some aspects of the disclosure, the third diagnostic model 570 includes a knowledge based expert system that combines both contextual domain knowledge and data-driven training to improve diagnostic accuracy and efficiency. In some aspects of the disclosure, the training phase of the third diagnostic model 570 includes generating a knowledge base that encodes expert knowledge in diagnosing PLN. The expert knowledge is obtained by interviewing medical professionals specializing in PLN to capture their diagnostic reasoning, differential diagnoses, and treatment strategies. The expert knowledge may also be gathered by analyzing (using either human experts or machine learning models) medical textbooks, research papers, documented cases (data) and guidelines for evidence-based practice. The acquired knowledge is appropriately structured and encoded for the training to be performed efficiently. For example, in some aspects of the disclosure, the knowledge about relationships between symptoms, findings, and diagnoses can be captured using production rules such as "IF-THEN" statements. In other aspects of the disclosure, the knowledge may be encoded as a Bayesian networks, which represents causal relationships between factors using probability. This permits the third diagnostic model 570 to consider the likelihood of different diagnoses based on various combinations of symptoms and test results. In yet other aspects of the disclosure, a semantic network may be used to connect symptoms, diseases, and tests through directed links, creating a web of interconnected knowledge that facilitates efficient information retrieval and inference.

In some aspects of the disclosure, the expert knowledge defines general rules and guidelines for the knowledge base, which are then refined using patient medical history and test data to train the third diagnostic model 570 to provide further diagnostic guidance and evaluation. The data is collected from diverse data sources, including electronic medical records, laboratory test results, clinical signs, and medication histories. The data is cleaned and validated, any missing or inconsistent values are addressed, and potential biases are removed before using the data for training. Data mining techniques like association rule mining and clustering are employed to identify hidden patterns and correlations within the data. This data mining analysis uncovers associations between specific symptom combinations and diagnoses, or reveal risk factors and disease progression patterns. The patterns and insights extracted using data mining are then used to refine the existing rules in the knowledge base. The data mining techniques used for training the third diagnostic model 570 automatically discover previously unknown relationships between symptoms and diagnoses, not readily apparent from expert knowledge alone. This allows the third diagnostic model 570 to stay relevant and adapt to evolving disease patterns and emerging medical literature.

In some aspects of the disclosure, the third diagnostic model 570 integrates probabilistic reasoning techniques with a rules-based engine to account for data variability and incomplete information. This enables the diagnostic system 500 to provide confidence levels associated with diagnoses, enhancing transparency and guiding further investigative steps. The third diagnostic model 570 employs an inference engine that translates the knowledge base and the responses to the queries from the user interface 560 into actionable insights. In some aspects of the disclosure, the inference engine includes both forward chaining and backward chaining inference strategies.

Forward chaining starts with known facts (symptoms, test results) and applies the rules iteratively to infer additional facts and ultimately reach a diagnosis/conclusion. Backward chaining works backward from a suspected diagnosis/conclusion, identifying supporting evidence and tests needed to confirm or refute it. Backwards chaining is especially useful to identify additional information that is needed to support a diagnosis or conclusion, and query the veterinarian, using the graphical user interface 560, to provide the additional information. The user interface 560 of the diagnostic system 500 permits the veterinarian to not only input patient symptoms but to also assign relative weights to their severity or duration, providing the diagnostic system 500 with richer context.

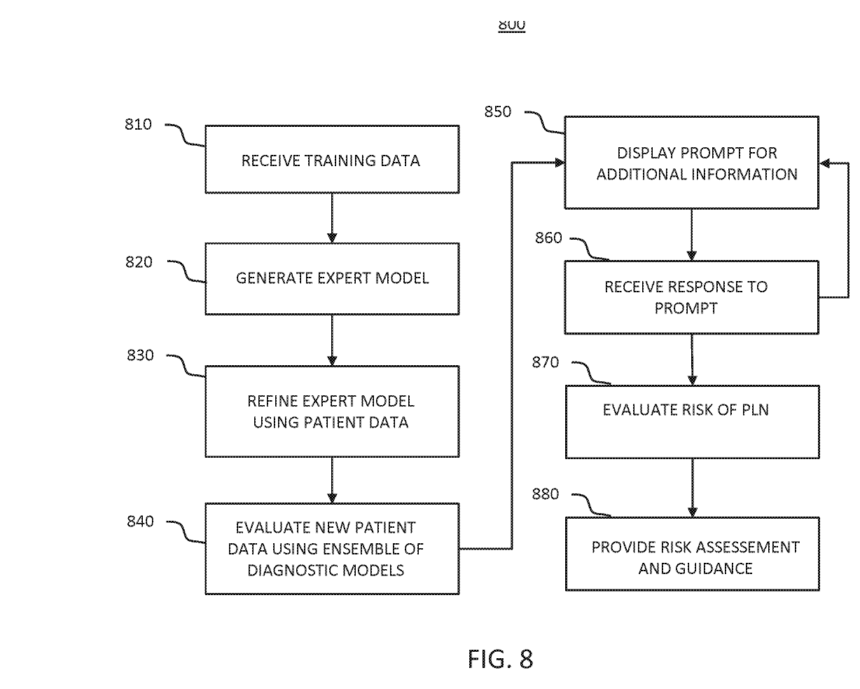

FIGS. 5 and 8 show a flowchart of a method 800 for training and using an interactive knowledge based model for assessing PLN risk, according to various aspects of the disclosure. In step 810, data for training the knowledge based model is obtained. The training data includes both domain knowledge and clinical/laboratory patient data. The domain knowledge is obtained, for example, from human experts.

In step 820, an expert model is generated using the obtained domain knowledge. The expert model may include rules-based inference, knowledge graphs, semantic ontologies, predicate logic or other techniques, known in the art, for representing domain knowledge.

In step 830, the expert model is refined using the clinical/laboratory patient data. This refinement could include adjusting the logic defined in the expert model, assigning weights to the various logic branches, adjusting the connections between various entities in the model etc.

In step 840, new patient data is received and assessed using the ensemble of diagnostic models 520. If the ensemble of diagnostic models indicates a potential risk of PLN, in steps 850 and 860, an interactive user interface is used to present a clinician with a sequence of prompts and receive responses to the sequence of prompts, to acquire additional data to assess the risk of PLN. Each new prompt presented in step 850 may be based on the response received to a previous prompt in step 860. Steps 850 and 860 may be repeated, as appropriate, to acquire the additional information required by the knowledge based model. In step 870, the risk of PLN is determined based on the acquired additional data. In step 880, the user interface is used to provide the clinician with an assessment of the risk for PLN on the new patient data and guidance for further testing and treatment.

In some aspects of the disclosure, the diagnostic system 500 provides differential diagnosis suggestions, presenting a list of possible diagnoses based on the entered information, considering the patient's medical history and test results. In some aspects of the disclosure, the diagnostic system 500 provides evidence-based recommendations, suggesting further tests or investigations based on the third diagnostic model’s 570 assessment and current clinical guidelines. In some aspects of the disclosure, the diagnostic system 500 provides explanatory reasoning for the diagnostic model’s conclusions and guidance, showing the reasoning chain behind the suggested diagnoses and tests, promoting transparency and trust with the veterinarian.

In some aspects of the disclosure, the diagnostic system 500, including the ensemble of diagnostic models 520 and the third diagnostic model 570, is continually evaluated and refined using anonymized patient data to assess its accuracy in clinical settings. Validation of the diagnostic system 500 is also performed based on feedback from medical professionals using the diagnostic system 500, to identify areas for improvement and ensure its practical utility. In some aspects of the disclosure, the knowledge base is regularly updated to incorporate new medical knowledge from medical experts, research, guidelines updates, and emerging disease patterns.

Various aspects of the diagnostic system 500 may be realized by software, or more precisely, an application program running on a microprocessor, or by firmware or hardware implementing the program on the POC terminal and/or the software servers POC. The diagnostic system 500 may include one or more memories which store various data and program modules associated with the diagnostic methods.

It is important to note that the machine learning and diagnostic algorithms described herein do not represent a computer application of the way humans perform diagnoses. Humans interpret new data in the context of everything else they have previously learned. In stark contrast to mental diagnostic processes, artificial intelligence algorithms, and specifically, the machine learning (ML) algorithms described herein, analyze massive data sets to identify patterns and correlations, without understanding any of the data they are processing. This process is fundamentally different from the mental process performed by a veterinarian. Furthermore, the large amounts of data required to the train the machine learning models, and the complexity of the trained models, make it impossible for the algorithms described herein to be performed merely in the human mind.

Accordingly, it should now be understood that some aspects of the present disclosure are directed to machine learning methods and systems for selecting and training diagnostic models for assessing the risk of PLN in patients.

It will be understood that various modifications may be made to the aspects and features disclosed herein. Therefore, the above description should not be construed as limiting, but merely as exemplifications of various aspects and features. Those skilled in the art will envision other modifications within the scope and spirit of the claims appended thereto.

What is claimed is:

1. A method for identifying the risk of protein losing nephropathy (PLN), comprising:
receiving new patient data;
receiving a machine-learning based diagnostic model and a knowledge based diagnostic model;
determining whether the machine-learning based diagnostic model indicates a risk for PLN for the new patient data; and
in a case where the machine-learning based diagnostic model indicates a risk for PLN for the new patient data, assessing the risk of PLN using the knowledge based diagnostic model.

2. The method according to claim 1, wherein the machine-learning based diagnostic model includes one or more of a decision tree model, a random forest model, a neural network, a support vector machine, or a nearest neighbor classifier.

3. The method according to claim 1, further comprising:
receiving first training data; and
training the machine-learning based diagnostic model by:
selecting at least a first machine learning model and a second machine learning model, wherein the second machine learning model is different from the first machine learning model;
training the first machine learning model using a first subset of the received first training data to generate a first diagnostic model;
training the second machine learning model using a second subset of the received first training data to generate a second diagnostic model; and
combining the first diagnostic model and the second diagnostic model into an ensemble diagnostic model to be used as the machine-learning based diagnostic model,
wherein the risk for PLN for the new patient data is determined based on an output of the ensemble diagnostic model.

4. The method according to claim 3, wherein it is determined that the ensemble diagnostic model indicates a risk for PLN for the new patient data in a case where the outputs of either the first diagnostic model or the second diagnostic model indicate the risk for PLN for the new patient data.

5. The method according to claim 1, further comprising:
receiving second training data; and
training the knowledge based diagnostic model by:
generating an expert model including a plurality of rules based on domain knowledge included in the received second medical training data; and
refining the plurality of rules in the expert model based on patient data included in the received second medical training data.

6. The method according to claim 1, wherein the knowledge based diagnostic model includes one or more of a rule based model or a data driven machine learning model.

7. The method according to claim 1,
wherein assessing the risk of PLN using the knowledge based diagnostic model further includes:
displaying, on a user interface, a series of prompts to receive further patient medical data; and
assessing the risk of PLN based the received further patient medical data in response to the series of prompts.

8. The method according to claim 7, wherein each subsequent prompt of the series of prompts is dynamically selected using the knowledge based diagnostic model based on a response to a preceding prompt of the series of prompts.

9. The method according to claim 1,
wherein the knowledge based diagnostic model is an interactive model, and
wherein assessing the risk of PLN includes interacting with a user using the knowledge based diagnostic model to provide guidance on diagnosis and treatment of PLN.

10. The method according to claim 1, further comprising analyzing a patient sample with an analyzer and determining the new patient data based at least in part on the analyzed patient sample.

11. The method of claim 10, wherein analyzing the patient sample comprises passing a blood sample through a cuvette and wherein the analyzer is a flow cytometer.

12. The method of claim 1, further comprising, in response to indicating a risk of PLN, administering an angiotensin converting enzyme.

13. A diagnostic system for assessing the risk of protein losing nephropathy (PLN), the system comprising:
a memory configured to store instructions;
an analyzer configured to analyze a patient sample; and
a processor communicatively connected to the memory and the analyzer and configured to execute the stored instructions to:
receive new patient data from the analyzer;
receive a machine-learning based diagnostic model;
determine whether the machine-learning based diagnostic model indicates a risk for PLN for the new patient data; and
in a case where the machine-learning based diagnostic model indicates a risk for PLN for the new patient data, assess the risk of PLN.

14. The system according to claim 13, wherein the analyzer is a flow cytometer comprising a cuvette and one or more sensors.

15. The system according to claim 13,
wherein the processor is further configured to execute the stored instructions to:
receive first training data; and
train the machine-learning based diagnostic model by:
selecting at least a first machine learning model and a second machine learning model, wherein the second machine learning model is different from the first machine learning model;
training the first machine learning model using a first subset of the received first training data to generate a first diagnostic model;
training the second machine learning model using a second subset of the received first training data to generate a second diagnostic model; and
combining the first diagnostic model and the second diagnostic model into an ensemble diagnostic model to be used as the machine-learning based diagnostic model, and
wherein the risk for PLN for the new patient data is determined based on an output of the ensemble diagnostic model.

16. The system according to claim 15, wherein it is determined that the ensemble diagnostic model indicates a risk for PLN for the new patient data in a case where the outputs of either the first diagnostic model or the second diagnostic model indicate the risk for PLN for the new patient data.

17. The system according to claim 13, wherein the processor is further configured to execute the stored instructions to:
receive second training data; and
train a knowledge based diagnostic model by:
generating an expert model including a plurality of rules based on domain knowledge included in the received second medical training data; and
refining the plurality of rules in the expert model based on patient data included in the received second medical training data.

18. The system according to claim 13, wherein the patient data comprises cell classifications.

19. The system according to claim 13,
wherein the risk of PLN is assessed using a knowledge based diagnostic model by:
displaying, on a user interface, a series of prompts to receive further patient medical data; and
assessing the risk of PLN based the received further patient medical data in response to the series of prompts.

20. The system according to claim 19, wherein each subsequent prompt of the series of prompts is dynamically selected using the knowledge based diagnostic model based on a response to a preceding prompt of the series of prompts.
